(19) United States
(12) Patent Application Publication
(10) Pub. No.: US 2025/0163591 A1
(43) Pub. Date: May 22, 2025

(54) Systems and Methods for Generating eFuels and Platform Chemicals from Carbon Based Fuel Combustion Sources

(71) Applicant: CARBONQUEST, INC., Spokane Valley, WA (US)

(72) Inventor: William A. Fuglevand, Spokane Valley, WA (US)

(73) Assignee: CARBONQUEST, INC., Spokane Valley, WA (US)

(21) Appl. No.: 18/832,927

(22) PCT Filed: Jan. 30, 2023
(86) PCT No.: PCT/US23/11863

§ 371 Date: Jul. 25, 2024

(51) Int. Cl. C25B 3/26; B01D 53/26; C01B 3/34; C07C 51/15; C10J 3/60; C25B 1/23; C25B 3/07; C25B 9/09; C25B 9/19; C25B 11/032; C25B 15/08

(52) U.S. Cl. CPC C25B 3/26; B01D 53/26; C01B 3/34; C07C 51/15; C10J 3/60; C25B 1/23; C25B 3/07; C25B 9/09; C25B 9/19; C25B 11/032; C25B 15/085; B01D 2256/20; B01D 2256/22; B01D 2257/102; B01D 2257/80; B01D 2258/0283; C01B 2203/0238

(57) ABSTRACT

Systems for generating eFuels from carbon based fuel combustion flue gas are provided. Systems for generating platform chemicals from carbon based fuel combustion flue gas are also provided. Methods for generating platform chemicals from carbon based fuel combustion flue gas are also provided.

### CROSS REFERENCE TO RELATED APPLICATION

This application claims priority to and the benefit of U.S. Provisional Patent Application Ser. No. 63/305,199 filed Jan. 31, 2022, entitled “CO₂ Separation from Combustion Streams and Conversion of CO₂ Separated from Combustion Streams”, the entirety of which is incorporated by reference herein.

### TECHNICAL FIELD

The present disclosure relates to CO₂ separation from combustion streams as well as CO₂ purification and conversion to eFuels and/or platform chemicals. The systems and processes of the present disclosure also provide for the use of the eFuels and/or platform chemicals as or from CO₂ sequestration materials.

### BACKGROUND

CO₂ is typically generated in substantial amounts as a combustion product of Air and carbonaceous fuel. The combustion product is not pure CO₂, but rather a mixture of other compounds as well. These other compounds can include global warming compounds, but none are present at the level of concentration of CO₂. Separating CO₂ from these other compounds, sequestering the separated CO₂ and/or using the CO₂ to form other compounds presents a significant challenge. Increasing reliance on fossil fuels for energy has led to an alarming rise in CO₂ concentration in the atmosphere which now exceeds 406 PPM. The International Energy Agency reports that anthropogenic emissions of CO₂ from fossil fuel combustion represent the largest portion of overall emissions. Growing concern for Climate Change has motivated international efforts to enforce regulations to combat global warming as agreed to at the Paris Climate Change Conference of 2015. Consensus is to reduce CO₂ emissions to less than 1000 GT equating to less than 2° C. atmospheric temperature rise. This daunting goal can only be reached by moving towards greener more efficient technologies including large scale incorporation of renewable energy sources. In addition, carbon capture (CCS) and sequestration is now proven technology which can contribute to nearly 12% of the Paris goals. The present disclosure provides systems and methods that overcome some of these challenges.

### SUMMARY

Systems for generating eFuels from carbon based fuel combustion flue gas are provided. The systems can include: a carbon based fuel combustion source; one or more components configured to receive flue gas from the carbon based fuel combustion source, the one or more components configured to dry, separate N₂ from CO₂, and purify the CO₂ to generate purified CO₂; and an eFuel generating component configured to produce an eFuel from the purified CO₂.

Systems for generating platform chemicals from carbon based fuel combustion flue gas are also provided. The systems can include: a carbon based fuel combustion source; one or more components configured to receive flue gas from the carbon based fuel combustion source, the one or more components configured to dry, separate N₂ from CO₂, and purify the CO₂ to generate purified CO₂; and a platform generating component configured to produce at least one platform chemical from the purified CO₂.

Methods for generating platform chemicals from carbon based fuel combustion flue gas are provided, the method comprising: receiving at least a portion of flue gas from a carbon based fuel combustion source; one or more of drying, separating N₂ from CO₂, and/or purifying the flue gas source to provide purified CO₂; and generating at least one platform chemical from the purified CO₂.

### DRAWINGS

Embodiments of the disclosure are described below with reference to the following accompanying drawings.

FIG. 1 is a depiction of a system and process for producing eFuel according to an embodiment of the disclosure.

FIG. 2 is a depiction of a CO₂ electrolysis cell according to an embodiment of the disclosure.

FIG. 3 is a depiction of an electrolysis system and process for producing and purifying CO according to an embodiment of the disclosure.

FIG. 4 is an electrolysis system and downstream process for forming an eFuel from CO₂ according to an embodiment of the disclosure.

FIG. 5 is a depiction of a CO₂ electrolysis cell according to an embodiment of the disclosure.

FIG. 6 is a depiction of a CO₂ electrolysis cell according to an embodiment of the disclosure.

FIG. 7 is a depiction of a CO₂ electrolysis cell according to an embodiment of the disclosure.

FIG. 8 is a system and process for forming platform molecules from CO₂ according to an embodiment of the disclosure.

FIG. 9 is a system and process for forming an eFuel from CO₂ according to an embodiment of the disclosure.

FIG. 10 is a system and process for forming an example eFuel from CO₂ according to an embodiment of the disclosure.

FIG. 11 is a system and process for forming another example eFuel from CO₂ according to an embodiment of the disclosure.

FIG. 12 is a system and process for forming another example eFuel from CO₂ according to an embodiment of the disclosure.

### DESCRIPTION

Electrolysis of CO₂ can be used to provide eFuels and production starting materials, eliminating dependency on traditional fossil fuels while at the same time removing significant global warming molecules from the atmosphere. Together with hydrogen (which can be provided renewably), carbon monoxide from the electrolysis of CO₂ captured from combustion can be processed to provide eFuels and high value chemicals consistent with world goals in sustainability.

Separating, purifying, liquefying, and/or storing CO₂ from building flue gas has been disclosed in published U.S. patent application Ser. No. 16/862,006 entitled “Building Emission Processing and/or Sequestration Systems and Methods”, U.S. Patent Application Publication No. US 2020/0340665 A1 published Oct. 29, 2020. These systems and processes can be improved to encompass methods of CO₂ conversion for purposes of downstream processing into value added eFuels and starting materials such as platform chemicals. This can include direct conversion of CO₂ and renewable hydrogen into liquid or gas products, or indirect conversion of CO₂ into CO and mixed with renewable hydrogen known as: “renewable syngas” (xCO+yH₂).

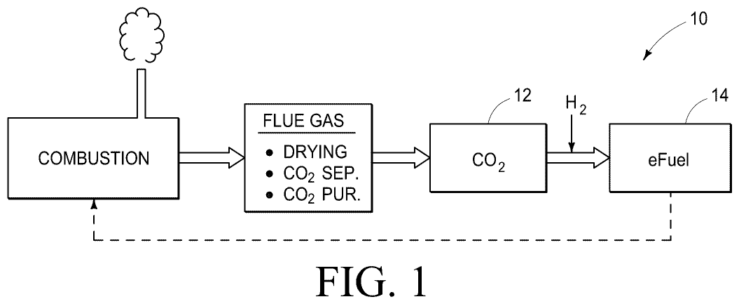

Embodiments of the systems and methods of the present disclosure will be described with reference to FIGS. 1-12. Referring first to FIG. 1, an overall schematic eFuel production process 10 is shown that incorporates portions of published U.S. patent application Ser. No. 16/862,006 entitled “Building Emission Processing and/or Sequestration Systems and Methods”, U.S. Patent Application Publication No. US 2020/0340665 A1 published Oct. 29, 2020. In accordance with example implementations, combustion can be from a residential, commercial, or industrial process, and flue gas during combustion is created. That flue gas can be processed in accordance with the above-referenced patent application to form a stream of CO₂ 12. This stream 12 can have a specific CO₂ concentration minimum and a specific water maximum. This CO₂ can be reacted with H₂ to form an eFuel 14.

eFuels can be any form of fuel derived from captured CO₂ and renewable H₂ and can be utilized to generate energy. These eFuels typically can be processed from syngas (carbon monoxide and hydrogen) to produce both oxygenated and non-oxygenated carbon compounds.

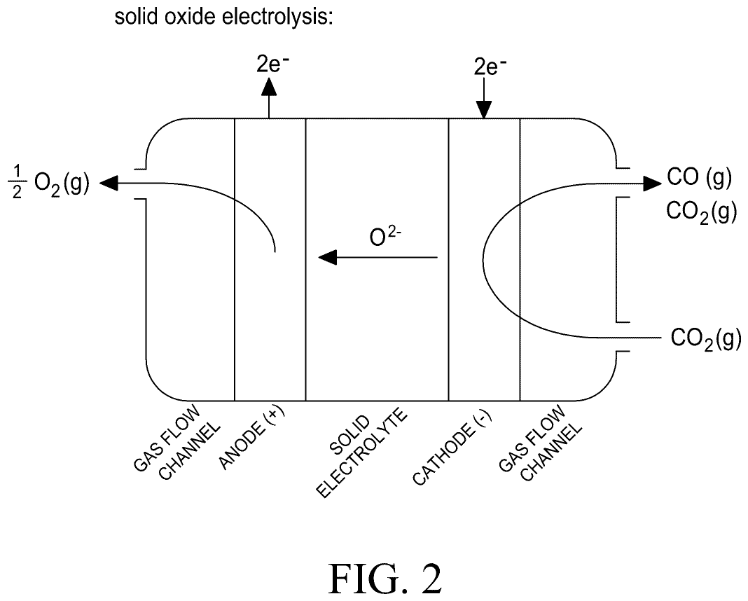

Referring next to FIG. 2, a detailed view of solid oxide electrolysis is shown. Utilizing DC electrical energy and a solid electrolyte, CO₂ can be chemically reduced to CO and O₂ within the solid oxide electrolysis cell.

The SOEC cell and method of FIG. 2 has a technology readiness level (TRL) of 8, while the other cells and methods described herein range from TRL=1 to TRL=4. FIG. 2 depicts a high temperature Solid Oxide electrochemical cell (SOEC) for DRY conversion of carbon dioxide to Carbon Monoxide and Oxygen. The SOEC uses a ceramic electrolyte which transfers the oxygen ion (O⁻⁻). Ionic conductivity generally increases exponentially with temperature.

Commonly used electrolytes include but are not limited to: stabilized zirconias (with yttria and scandia), and/or doped cerias (with gadolinia and samaria). Two electrodes, the Anode and Cathode are in contact with the solid electrolyte. Electrodes are normally composites of Nickel which provide both electrical conductivity and catalyst activation. This particular cell can operate endothermically and can be maintained at an appropriate temperature. Faradaic efficiency for CO production in this cell can approach 100%. Current densities in excess of 750 mA/cm² are common. The electrochemical equations for electrolysis are:

Anode ½O₂⁻⁻>>½O₂+2e⁻

Cathode CO₂+2e⁻>>CO+½O₂⁻⁻

The electrons can be provided by an external power supply which is preferably a renewable power supply.

The Enthalpy of formation for carbon monoxide at standard conditions is −110 kj/mol. For the overall reaction above we have: CO₂>>CO+½O₂. Thus, the applied enthalpy is: −393>>−110−0 or, Delta H going to the right is: 283 kj/mol, which is the total energy required to produce one mol of carbon monoxide.

When converting CO₂ entirely with electricity, this electrolysis energy can be expressed in units of kWhr per cubic feet of CO, and in units of kWhr per normal cubic meter of CO, as shown below:

$$283 kj / mol CO = 0.0786 kWhr / mol CO = 0.0994 kWhr / {ft}^{3} .$$

$$CO = 3.39 kWhr / {Nm}^{3} CO ( IDEAL )$$

Thus at an estimated 60% electrolysis efficiency (realistic), we would need 5.65 kWhr of electrical energy to produce a standard cubic meter of CO.
(1 Nm³ of gas is equivalent to 34 standard cubic feet.)

The total energy delta H, however, can be made up with a combination of both heat and electricity, if desired.

Heat energy required to heat incoming gases is different from reaction heat discussed above. It is desired that the systems and processes of the present disclosure will transfer heat from output gases to input gases to raise overall efficiency of electrolysis. The design also takes advantage of Joule heat from ionic conduction through the electrolyte.

The electrochemical cell shown in FIG. 2 normally runs at a temperature ranging from 500° C. to 1000° C.

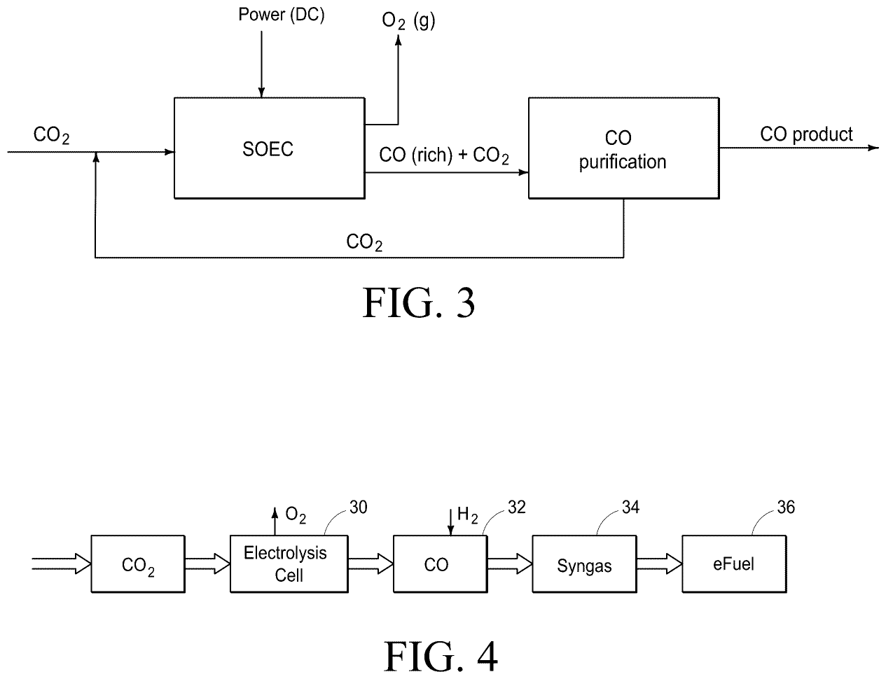

From FIG. 2, one can see that the output stream can contain some small fraction of unconverted CO₂. Accordingly, in the practical design, a purification section is added to separate the CO₂ for feed back into the process, while producing a high purity stream of output CO. In accordance with FIG. 3, the SOEC electrolysis product of CO and CO₂ can be further purified to high purity CO while returning CO₂ to the SOEC cell input. Accordingly, no CO₂ is emitted to the atmosphere.

Referring to FIG. 4, an example system and process for the conversion of CO₂ into CO and Syngas and then generally forming an eFuel is shown. As shown, at electrolysis component 30, CO can be generated. At component 32, H₂ can be added to the CO to form a Syngas.

In accordance with FIG. 4, the CO₂ can be provided from the process of FIG. 1, for example, in more than one stream but at least CO₂ can be provided in substantially pure form to a Solid Oxide electrochemical cell. Power can be provided to the cell, and CO₂ can be reduced to high concentration CO with a minor amount of CO₂ provided for final CO gas purification and to form a CO product which can be processed with hydrogen into an eFuel. Excess CO₂ separated during CO purification can be recycled to the Solid Oxide electrochemical cell input.

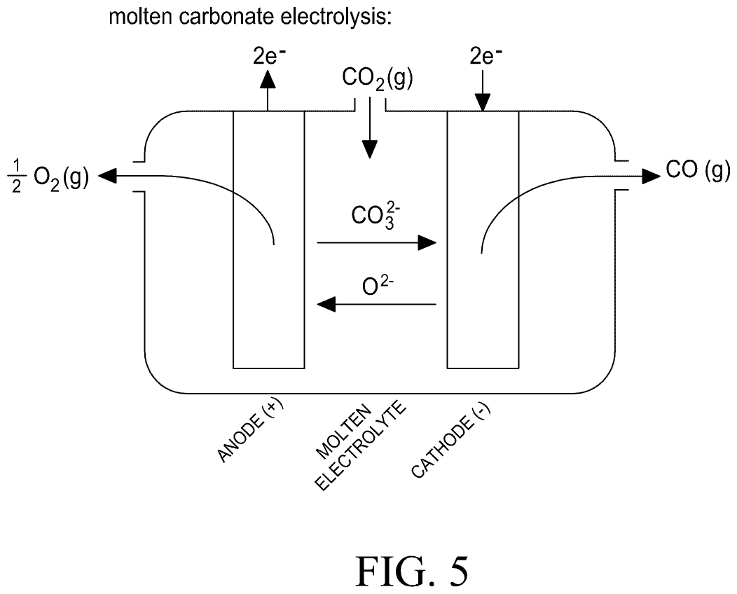

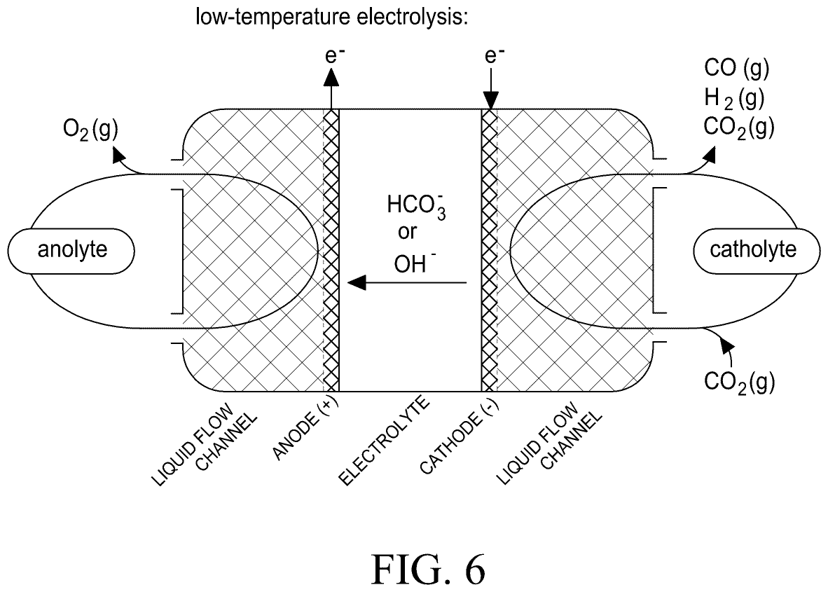

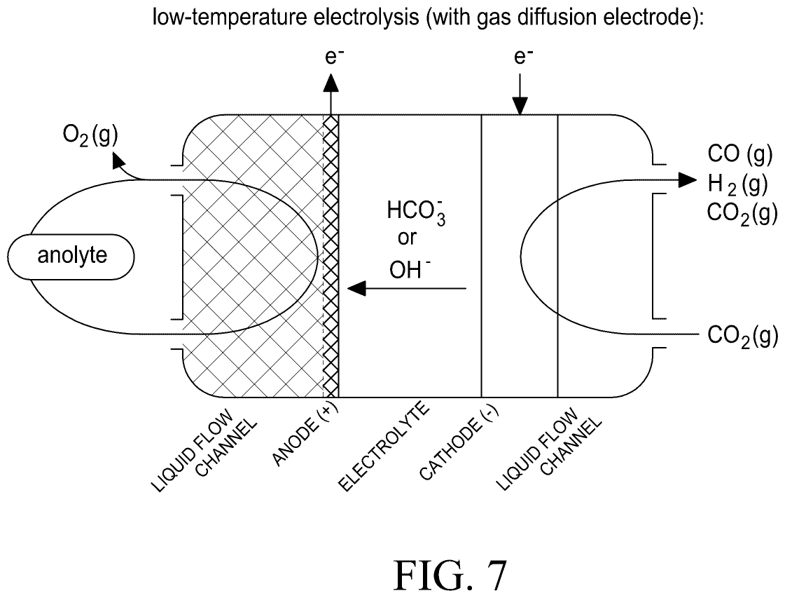

Several additional CO₂ to CO electrolysis cells in addition to the one shown in FIG. 2 are shown in FIGS. 5 to 7. These are: FIG. 5, molten carbonate electrolysis; FIG. 6, low temperature electrolysis; and FIG. 7, low temperature electrolysis with gas diffusion. These electrolysis components can share some functional elements such as two distinct electrodes in contact with an electrolyte (solid or liquid). These electrolytes are associated with the ions which they conduct, such as: protons, hydroxide ions, oxide ions, carbonate and bi-carbonate ions. With applied electricity, reduction takes place at the negative potential Cathode, and Oxidation takes place at the positive potential Anode.

Referring next to FIG. 5, an electrochemical cell and method is depicted where the electrolyte is a carbonate melt similar to the electrolyte of a molten carbonate electrolysis cell (MCEC) operating at 500° C. to 800° C. Components for the MCEC can include a combination Li₂O/LiCO₃ molten electrolyte with titanium Cathode and graphite Anode. Li₂CO₃ can be converted into Li₂O electrochemically at the Cathode thus increasing the oxide melt ratio allowing more CO₂ concentrated above the melt to be incorporated into the mixture. The MCEC can provide an output of high purity CO and/or it can utilize CO₂ streams diluted with some water vapor. Faradaic efficiency for CO production in this cell can approach 100%. Current densities as high as 3 A/cm² have been demonstrated in polarization measurements.

Referring next to FIG. 6, an electrochemical cell and method is depicted as a low temperature electrochemical cell (low temperature electrolysis H-cell) where reduction and oxidation occur in liquid electrolyte solutions, catholyte and anolyte respectively. Electrolytes can include solid membrane dividers such as a proton exchange membrane (PEM) or anion exchange membrane (AEM), and aqueous solutions of KHCO₃, or a combination. Most low temperature electrolysis cells operate in alkaline conditions. Each cell uses an H-cell configuration whereby both electrodes are immersed in respective electrolyte solutions (Anolyte and Catholyte). FIG. 6 depicts a configuration with an anion exchange membrane (AEM) divider. The anode catalyst is typically IrO₂, and the cathode catalyst being gold and silver nanoparticles, with appropriate supporting material for selectivity and stability. (i.e., Carbon nanotubes).

Referring next to FIG. 7, an electrochemical cell and method is depicted as a low temperature electrochemical cell with gas diffusion where reduction occurs in aqueous solutions. This cell differs from the H-cell in FIG. 6 by addition of gas diffusion electrodes to overcome mass balance limitations. These cells may have beneficial application in direct CO₂ to chemical production as for: formic acid, ethylene, ethanol, etc. In addition, the CO pathway to key chemicals may also be relevant for low temperature electrolysis cells. Neither of these routes is possible with high temperature electrochemical cells.

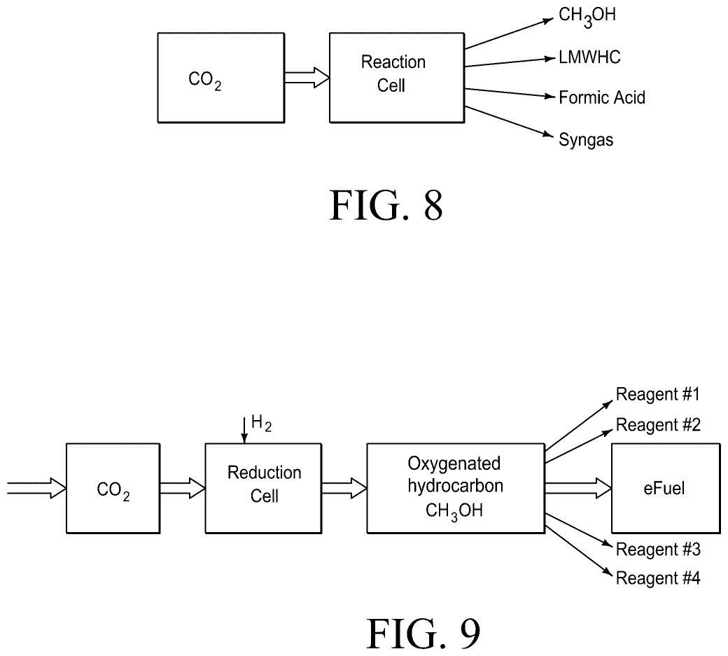

Referring next to FIGS. 8-12, systems and processes are provided for utilizing CO₂ separated and purified from combustion sources as the carbon source, for not only eFuels, but for many other carbon-based materials, such as platform chemicals and application specific chemical reagents. FIG. 8 depicts a generic system and process for preparing platform molecules and/or mixtures from CO₂. For example, CO₂ obtained from flue gas pursuant to the techniques referenced herein can be processed to form CH₃OH, low molecular weight hydrocarbons, formic acid, and/or syngas.

FIG. 9 is just one example that provides for the selective hydrogenation of CO₂ to form methanol, a common platform chemical, which can be converted to eFuels and many other application specific reagents, including polymer precursors.

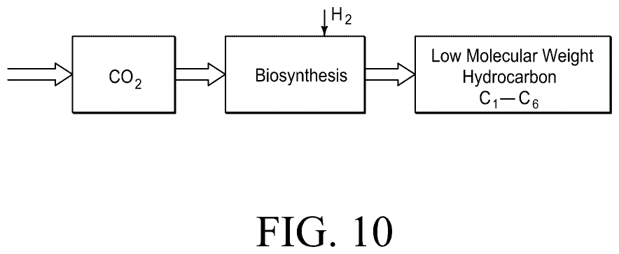

FIG. 10 provides an example system and process for the direct conversion of CO₂ via biosynthesis to low molecular weight hydrocarbons which can form the starting materials for a myriad of industrial compounds which, heretofore, have been provided from the petroleum industry.

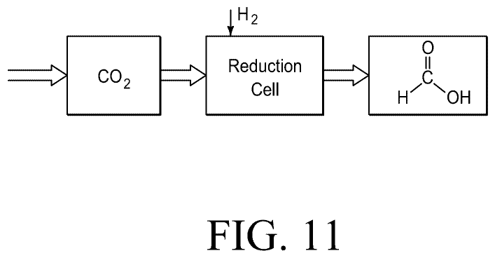

FIG. 11 provides an example system and process for the conversion of CO₂ via electrochemical reduction to the formate ion, and finally to formic acid, which is another valuable oxygenated hydrocarbon and sustainable green platform chemical, also used as a hydrogen carrier.

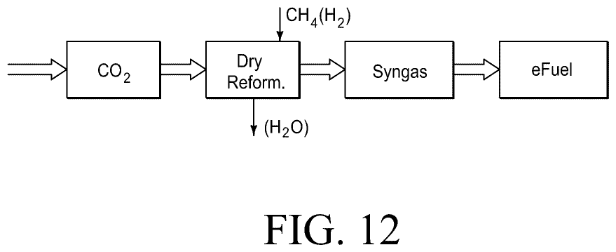

Additionally, FIG. 12 provides an example system and process for the conversion of CO₂ via dry reformation to syngas, using methane (CH₄), or hydrogen which results in surplus production of water.

Beyond sequestration is the need to produce “sustainable” fuels of the future from captured CO₂ and green hydrogen. These eFuels can be carbon neutral and can greatly reduce the need for traditional fossil fuels, fossil fuel processing, and/or fossil fuel by-products. In addition to eFuels, high value platform chemicals (low molecular weight hydrocarbons, methanol, and/or formic acid) can also be produced from captured CO₂, green hydrogen, and in specific cases such as formic acid.

As described herein, electrochemical and direct catalytic reactions can be used to form these eFuels and platform chemicals. Since energy is required, renewable sources of energy such as solar, wind, tiadal, and geothermal are preferred. Thus, candidate processes include: electrolysis of CO₂, electro-reduction, photocatalytic, selective hydrogenation, and biocatalytic conversion. Since syngas (xCO+yH₂) can be used as a reagent for production higher molecular weight (C4-C16) fuels via the proven Fischer Tropsch process, electrolysis of CO₂ to produce CO thus becomes a favored CO₂ front end conversion method. Highly efficient CO₂ conversion methods will require novel combinations of processes and materials.

Additionally, a relatively new branch of Materials Science called Reticular Chemistry has been shown to produce robust molecular framework materials with enormous surface areas (upwards to 10,000 square meters per gram) with high volume uniform pore sites, greatly exceeding the capacities of natural and synthetic silicate materials prevalent today. Design of these framework materials offer vast possibilities of chemistries for node, linkers, and interconnections. In addition, these materials can be geometrically optimized and/or specifically functionalized pre and post syntheses to impart desired properties including improved catalytic properties.

It is envisioned that framework materials will revolutionize solid adsorption gas separation and/or conversion processes of the future. Desirable characteristics of candidate molecular framework materials for CO₂ separation from flue gas are: selectivity, capacity, durability, scalability, hydrophobicity, physisorption, stable cyclic operation in temperatures up to 150° C., and in pressures from 10 mbar up to 7 bar. In addition, the pores within framework materials can be configured for catalytic, enzymatic, and ionic transfer functions. Framework materials can also be included in structured Mixed-Matrix-Membranes (MMM's). From these perspectives designer molecular framework materials will become essential to highly efficient CO₂ separation and conversion processes.

1: A system for generating eFuels from carbon based fuel combustion flue gas, the system comprising:
a carbon based fuel combustion source;
one or more components configured to receive flue gas from the carbon based fuel combustion source, the one or more components configured to dry the flue gas to remove H₂O, then separate N₂ from CO₂ to remove N₂ from the N₂ and CO₂ and purify the separated CO₂ to generate purified CO₂; and
an eFuel generating component configured to produce an eFuel from the purified CO₂.

2: The system of claim 1 wherein the eFuel generating component comprises a solid oxide electrochemical cell.

3: The system of claim 2 wherein the solid oxide electrochemical cell comprises a solid electrolyte.

4: The system of claim 2 wherein the solid oxide electrochemical cell comprises a molten electrolyte.

5: The system of claim 2 wherein the eFuel generating component further comprises a CO purification component.

6: The system of claim 2 wherein the eFuel generating component further comprises a syngas generating component.

7: A system for generating platform chemicals from carbon based fuel combustion flue gas, the system comprising:
a carbon based fuel combustion source;
one or more components configured to receive flue gas from the carbon based fuel combustion source, the one or more components configured to dry, the flue gas to remove H₂O, then separate N₂ from CO₂ to remove N₂ from the N₂ and CO₂ and purify the separated CO₂ to generate purified CO₂; and
a platform generating component configured to produce at least one platform chemical from the purified CO₂.

8: The system of claim 7 wherein the platform chemical is one or more of syngas, CH₃OH, LMWHC, and/or formic acid.

9: The system of claim 8 wherein the platform generating component is a reduction cell.

10: The system of claim 9 wherein the platform chemical is one or both of CH₃OH or formic acid.

11: The system of claim 8 wherein the platform generating component is a biosynthesis cell and the platform chemical is a LMWHC.

12: The system of claim 7 wherein the platform generating component is configured as a dry reformation component and the platform chemical is syngas.

13: A method for generating platform chemicals from carbon based fuel combustion flue gas, the method comprising:
receiving at least a portion of flue gas from a carbon based fuel combustion source;
drying the flue gas to remove H₂O;
separating N₂ from CO₂, of the dried flue gas to remove N₂;
purifying the CO₂ separated from the N₂ to provide purified CO₂; and
generating at least one platform chemical from the purified CO₂.

14: The method of claim 13 wherein the platform chemical is an eFuel.

15: The method of claim 14 wherein the generating comprises electrolysis using one or more of a solid electrolyte, a molten electrolyte, low temperature electrolysis, and/or low temperature electrolysis with a gas diffusion electrode.

16: The method of claim 15 wherein the electrolysis generates CO.

17: The method of claim 16 wherein the method further comprises mixing the CO with H₂ to form syngas.

18: The method of claim 13 wherein the generating the at least one platform chemical comprises reducing CO₂ to form CH₃OH.

19: The method of claim 13 wherein the generating the at least one platform chemical comprises forming LMWHC from the biosynthesis of CO₂.

20: The method of claim 13 wherein the generating the at least one platform chemical comprises reducing the CO₂ to form formic acid.

21: The method of claim 13 wherein the generating the at least one platform chemical comprises dry reformation of the CO₂ to form syngas.
